(19) United States
(12) Patent Application Publication
(10) Pub. No.: US 2017/0100327 A1
(43) Pub. Date: Apr. 13, 2017

(54) PHARMACEUTICAL COMPOSITION WITH IONICALLY CROSSLINKED POLYMER ENCAPSULATION OF ACTIVE INGREDIENT

(71) Applicant: CURE Pharmaceutical Corporation, Oxnard, CA (US)

(72) Inventor: Eric Allen, Camarillo, CA (US)

(21) Appl. No.: 15/315,264

(22) PCT Filed: May 29, 2015
(86) PCT No.: PCT/US15/33274

§ 371 Date: Nov. 30, 2016

Related U.S. Application Data
(60) Provisional application No. 62/005,481, filed on May 30, 2014.

(51) Int. Cl. A61K 9/00; A61K 31/357; A61K 9/50; A61K 31/4706

(52) U.S. Cl. CPC A61K 9/006; A61K 31/4706; A61K 31/357; A61K 9/5036; A61K 9/5089

(57) ABSTRACT

Compositions and thin films containing an encapsulated active pharmaceutical ingredient, as well as methods of manufacturing and using the same.

### PRIORITY APPLICATION

This application claims the benefit of U.S. Provisional Application Ser. No. 62/005,481, filed on 30 May 2014, which application is incorporated herein by reference in its entity.

### BACKGROUND OF THE INVENTION

Many active pharmaceutical ingredients taste bitter and thus are aversive to children as well as many adults. Because children are more bitter-sensitive than are adults, this creates problems with compliance, especially with children. Encapsulation of the medicine in pill or tablet form (an effective method for adults to avoid the unpleasant taste) is still problematic for children and some adults. This is so because many children and adults cannot or will not swallow solid dose forms. Better-tasting medications may enhance both adult and pediatric adherence to drug therapy. Sugars, acids, salt, and other substances reduce perceived bitterness of several pharmaceuticals, and although pleasant flavorings may help patients consume some medicines, they often are not effective in suppressing bitter tastes.

Additionally, several active pharmaceutical ingredients are not stable and undergo degradation during the manufacturing process of the finished dosage form. The instability can be due, e.g., to the presence of water, alone or in combination with elevated temperatures and/or the presence of oxygen.

### SUMMARY OF THE INVENTION

The present invention provides for a composition. The composition includes: (a) a first ionically-functionalized polymer; (b) at least one of (i) a polyionic compound and (ii) a second ionically-functionalized polymer, each independently having a charge opposite to that of the first ionically-functionalized polymer; (c) solvent; and (d) active ingredient.

The present invention provides for another composition. The composition includes: (A) a first solution that includes: (i) solvent; (ii) basic ionically-functionalized polymer; (iii) active ingredient; and (iv) optionally an acid; and (B) a second solution that includes: (i) solvent; (ii) acidic ionically-functionalized polymer; and (iii) optionally a base.

The present invention provides for another composition. The composition includes: (A) a first solution that includes: (i) aqueous solvent; (ii) basic ionically-functionalized polymer; (iii) active ingredient; and (iv) inorganic acid; and (B) a second solution that includes: (i) aqueous solvent; (ii) acidic ionically-functionalized polymer; and (iii) optionally an inorganic base.

The present invention also provides for a thin film manufactured from the composition.

The present invention also provides for a method of preparing an encapsulated active pharmaceutical ingredient. The method includes contacting a first solution that includes: (a) solvent; (b) first ionically-functionalized polymer; (c) active ingredient; and (d) optionally an acid or base; with a second solution that includes: (e) solvent; (f) at least one of (i) a polyionic compound and (ii) a second ionically-functionalized polymer, each having a charge opposite to that of the first ionically-functionalized polymer; and (g) optionally an acid or base.

The present invention also provides for a method of preparing an encapsulated active pharmaceutical ingredient. The method includes contacting a first solution that includes: (a) aqueous solvent; (b) basic ionically-functionalized polymer; (c) active ingredient; and (d) inorganic acid; and with a second solution that includes: (e) aqueous solvent; (f) acidic ionically-functionalized polymer; and (g) optionally an inorganic base.

The present invention also provides for a method that includes: (A) forming a first solution that includes: (i) dissolving chitosan in a mixture of water and acid, to form a first polymeric mixture; (ii) dissolving an active ingredient in the first polymeric mixture, to form the first solution; (B) forming a second solution that includes: (iii) dissolving iota-carrageenan sodium salt in water and optionally a base, to form the second solution; and (C) adding the first solution to the second solution, while blending or mixing, to form a mixture that includes solids suspended in a liquid; (D) separating the solids from the liquid; and (E) washing and drying the solids.

The present invention also provides for a thin film that includes: (a) a first ionically-functionalized polymer; (b) at least one of (i) a polyionic compound and (ii) a second ionically-functionalized polymer, each independently having a charge opposite to that of the first ionically-functionalized polymer; (c) solvent; (d) binder, (e) lipid & emulsifier, and (f) active ingredient.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a method for producing an encapsulated active pharmaceutical ingredient (API).

FIG. 2 illustrates a method for producing an encapsulated active pharmaceutical ingredient (API).

FIG. 3 illustrates a method for producing an encapsulated active pharmaceutical ingredient (API).

FIG. 4 illustrates a method for producing an encapsulated active pharmaceutical ingredient (API).

FIG. 5 illustrates a method for producing a thin film.

FIG. 6 illustrates a method for producing a thin film.

### DETAILED DESCRIPTION OF THE INVENTION

Reference will now be made in detail to certain claims of the invention, examples of which are illustrated in the accompanying drawings. While the invention will be described in conjunction with the enumerated claims, it will be understood that they are not intended to limit those claims. On the contrary, the invention is intended to cover all alternatives, modifications, and equivalents, which can be included within the scope of the invention as defined by the claims.

References in the specification to “one embodiment,” “an embodiment,” “an example embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

In this document, the terms “a,” “an,” or “the” are used to include one, or more than one, unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

“Oral thin film,” “OTF,” “oral dissolving film,” “oral drug strip,” “oral thin film,” “thin film,” “orally dissolvable film strip,” or “oral strip” refers to a product used to administer active ingredients via absorption in the mouth (buccally or sublingually), the stomach (gastrically), and/or via the small intestines (enterically). The OTF is edible and pharmaceutically acceptable. A film is prepared typically using hydrophilic polymers that rapidly dissolves on the tongue or buccal cavity, delivering the active ingredient to the systemic circulation via dissolution when contact with liquid is made. The OTF (or more appropriately “thin film” or “TF”) can also be used to adhere to mucosal tissue (e.g., at least one of mouth, nose, eye, vagina, and rectum), thereby locally delivering the active ingredient(s). As such, it is appreciated that those of skill in the art understand that reference to a thin film for use with mucosal tissue, such as nose, eye, vagina, and rectum, as an “oral thin film” or OTF is appropriate and acceptable.

The term “film” includes thin films and sheets, in any shape, including rectangular, square, or other desired shape. The films described herein may be any desired thickness and size such that it may be placed into the oral cavity of the user. For example, the films may have a relatively thin thickness of from about 0.1 to about 10 mils, or they may have a somewhat thicker thickness of from about 10 to about 30 mils. For some films, the thickness may be even larger, i.e., greater than about 30 mils. In addition, the term “film” includes single-layer compositions as well as multi-layer compositions, such as laminated films. The composition in its dried film form can effectively maintain a relatively uniform distribution of components through the application of controlled drying of the film. For example, the film can have no more than a 20%, 10%, 5%, or 1% variance of the active ingredient, per unit area of the film.

The substances can be selected in an amount such that a desired dissolution rate can be targeted. Upon contact with mucosal tissue (including, e.g., oral mucosa) the TF will completely dissolve within the desired period of time. The period of time will vary but in reference to the oral cavity, the period of time will typically be within about 30-300 seconds.

Dissolving films generally fall into three main classes: fast dissolving, moderate dissolving and slow dissolving. Fast dissolving films generally dissolve in about 1 second to about 30 seconds. Moderate dissolving films generally dissolve in about 1 to about 30 minutes, and slow dissolving films generally dissolve in more than 30 minutes.

The thin film can be manufactured in a manner, employing the ingredients described herein, such that any one or more of the desired pharmacokinetic metrics (e.g., dose, area under the curve, peak plasma concentration, dosing intervals, time to reach peak plasma concentration, clearance, bioavailability, etc.) are achieved. For example, the thin film can be manufactured such that the thin film provides for an immediate release (IR), controlled release (CR), modified release (MR), extended release (ER), or combination thereof, of active ingredient. This can be advantageous in those embodiments wherein multiple active ingredients are employed, each having different chemical and/or physical properties (e.g., pharmacokinetics, absorption kinetics, stability, solubility, bioavailability, etc.). The thin films described herein therefore possess the potential to allow the development of sensitive drug targets that may otherwise not be feasible in tablet or liquid formulations.

“Multiple” refers to two or more (e.g., 2, 3, 4, 5, 6, etc.).

“Solvent” refers to a substance capable of dissolving another substance (a solute), resulting in a solution. When one substance is dissolved into another, a solution is formed. This is opposed to the situation when the compounds are insoluble like sand in water. In solution, all of the ingredients are uniformly distributed at a molecular level and no residue remains. The mixing is referred to as miscibility, whereas the ability to dissolve one compound into another is known as solubility. However, in addition to mixing, both substances in the solution interact with each other. When something is dissolved, molecules of the solvent arrange themselves around molecules of the solute. Heat is involved and entropy is increased making the solution more thermodynamically stable than the solute alone. This arrangement is mediated by the respective chemical properties of the solvent and solute, such as hydrogen bonding, dipole moment and polarizability.

In particular reference to the thin films described herein, the solvent will typically dissolve, but may also suspend, the active ingredient and other substances present in the OTF. During the condensing step, much (if not all) of the solvent can be removed. However, any solvent remaining will become an integral part of the OTF.

As used herein, “ionically-functionalized polymer” refers to a polymer having one or more ionic functionalities, per monomer. The ionically-functionalized polymer can include basic polymers and/or acidic polymers.

The basic ionically-functionalized polymer can include amino (—NH₂) groups and/or quaternary ammonium cations (—NH₃⁺). Specifically, the basic ionically-functionalized polymer can include secondary ammonium groups, tertiary ammonium groups, and/or quaternary ammonium groups. Specific basic ionically-functionalized polymers include, e.g., zein, chitosan and polyquaternium.

The acidic ionically-functionalized polymer can include carboxylic acid (—CO₂H) groups, sulfate (—OSO₃H) groups, sulfonate (—SO₃H), phosphate (—OPO₃H₂) groups, and/or phosphonate (—PO₃H₂) groups. Specific acidic ionically-functionalized polymers include, e.g., pectin, xanthan gum, careageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya.

As used herein, “polyionic compound” refers to a compound having multiple ionic functionalities, per monomer. The polyionic compound can include, e.g., carboxylic acid (—CO₂H) or corresponding ester groups (such as —CO₂R, wherein, e.g., R is optionally substituted alkyl, optionally substituted aryl alkyl, or optionally substituted cycloalkyl alkyl) and/or phosphate (—OPO₃H₂) groups. In specific embodiments, the polyionic compound can be an acidic polyionic compound. Additionally, in further specific embodiments, the polyionic compound can be a polyanionic compound. Specific polyionic compounds include, e.g., citric acid, sodium triphosphate, malonic acid, malic acid, fumaric acid, succinic acid, glutaric acid, adipic acid, pimelic acid, and suberic acid. In specific embodiments, the polyionic compound includes one or more acidic non-polymeric crosslinkers.

As used herein, “polyanionic compound” refers to a compound having multiple anionic functionalities, per monomer, wherein an anion is a negatively charged ion.

As used herein, “lipid” refers to a group of naturally occurring molecules that include fats, waxes, sterols, fat-soluble vitamins (such as vitamins A, D, E, and K), monoglycerides, diglycerides, triglycerides, phospholipids, and others. The compounds are hydrophobic or amphiphilic small molecules. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits).

Although the term lipid is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol.

As used herein, “emulsifier” refers to a substance capable of forming or promoting an emulsion. An emulsion is a mixture of two or more liquids that are normally immiscible (nonmixable or unblendable). Emulsions are part of a more general class of two-phase systems of matter called colloids. Although the terms colloid and emulsion are sometimes used interchangeably, emulsion should be used when both the dispersed and the continuous phase are liquids. In an emulsion, one liquid (the dispersed phase) is dispersed in the other (the continuous phase). Examples of emulsions include vinaigrettes, milk, mayonnaise, and some cutting fluids for metal working. The photo-sensitive side of photographic film is an example of a colloid. In particular reference to the thin films described herein, the emulsifier promotes the separation of phases (e.g., aqueous and lipids), while allowing them to be mixed.

As used herein, “zein” refers to a class of prolamine protein found in maize (corn). It is usually manufactured as a powder from corn gluten meal. Pure zein is clear, odorless, tasteless, hard, water-insoluble, and edible. Zein is a basic ionically-functionalized polymer, having numerous proline (containing secondary amino) groups. This is so because when proline is bound as an amide in a peptide bond, its nitrogen is not bound to any hydrogen, meaning it cannot act as a hydrogen bond donor, but can be a hydrogen bond acceptor.

Specific lipids & emulsifiers include, e.g., glycerin, propylene glycol, and/or polyethylene glycol.

As used herein, “binder” refers to any material or substance that holds or draws other materials together to form a cohesive whole. Liquid binders are added to a dry substance in order to draw it together in such a way that it maintains a uniform consistency. Suitable binders include, e.g., pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, carboxymethyl cellulose (CMC), and polyethylene glycol.

The thin film described herein can optionally further include a mucoadhsesive agent. The mucoadhesive agent, when placed in the oral cavity in contact with the mucosa therein, adheres to the mucosa. The mucoadhesive agent is especially effective in transmucosal delivery of the active ingredient, as the mucoadhesive agent permits a close and extended contact of the composition with the mucosal surface by promoting adherence of the composition or drug to the mucosa, and facilitates the release of the active ingredient from the composition. The mucoadhesive agent can be a polymeric compound, such as a cellulose derivative but it may be also a natural gum, alginate, pectin, or such similar polymer. The concentration of the mucoadhesive agent in the coating, such as a powder matrix coating, may be adjusted to vary the length of time that the film adheres to the mucosa or to vary the adhesive forces generated between the film and mucosa. The mucoadhesive agent may adhere to oral mucosa or to mucosa or tissue in other parts of the body, including the mouth, nose, eyes, vagina, and rectum.

Mucoadhesive agents include, e.g., carboxymethylcellulose, polyvinyl alcohol, polyvinyl pyrrolidone (povidone), sodium alginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl acetate, dimenthylpolysiloxanes, polyoxyalkylene block copolymers, pectin, chitosan, carrageenan, xanthan gum, gellan gum, locust bean gum, and hydroxyethylmethacrylate copolymers.

“Encapsulated” refers to the enclosure of a first substance (e.g., active ingredient) by a second substance (e.g., matrix formed from the first ionically-functionalized polymer at least partially ionically crosslinked to the second ionically-functionalized polymer and polyionic compound). As described herein, the second substance can encapsulate the first substance. One advantage of the encapsulation of the thin films described herein is the ability to employ bitter substances (e.g., bitter active ingredients), while having the bitter flavor of those substances be at least partially masked. The encapsulation can be partial or complete.

As used herein, “crosslinked” or “cross-link” refers to a bond that links one polymer chain to another (or to the substance that has undergone crosslinking). They can be covalent bonds or ionic bonds (e.g., covalently cross-linked or ionically cross-linked, respectively). “Polymer chains” can refer to synthetic polymers or natural polymers (such as proteins). The term “cross-linking” refers to the use of cross-links to promote a difference in the polymers' physical properties.

When polymer chains are linked together by cross-links, they lose some of their ability to move as individual polymer chains. For example, a liquid polymer (such as resin or even melted cheese which contains protein polymers) (where the chains are freely flowing) can be turned into a “solid” or “gel” by cross-linking the chains together.

When a polymer is said to be “cross-linked,” it typically means that the entire bulk of the polymer has been exposed to the cross-linking method. The resulting modification of mechanical properties depends strongly on the cross-link density. Low cross-link densities decrease the viscosities of polymer melts. Intermediate cross-link densities transform gummy polymers into materials that have elastomeric properties and potentially high strengths. Very high cross-link densities can cause materials to become very rigid or glassy, such as phenol-formaldehyde materials.

Cross-links can be formed by chemical reactions that are initiated by heat, pressure, change in pH, or radiation. For example, mixing of an unpolymerized or partially polymerized resin with specific chemicals called crosslinking reagents results in a chemical reaction that forms cross-links. Cross-linking can also be induced in materials that are normally thermoplastic through exposure to a radiation source, such as electron beam exposure, gamma-radiation, or UV light. For example, electron beam processing is used to cross-link the C type of cross-linked polyethylene. Other types of cross-linked polyethylene are made by addition of peroxide during extruding (type A) or by addition of a cross-linking agent (e.g. vinylsilane) and a catalyst during extruding and then performing a post-extrusion curing.

Cross-links are the characteristic property of thermosetting plastic materials. In most cases, cross-linking is irreversible, and the resulting thermosetting material will degrade or burn if heated, without melting.

As used herein, “disperse” or “dispersed” refers to heterogeneous systems consisting of a mechanical mixture of particles contained within a continuous medium.

In particular reference to the thin films described herein, the emulsifier promotes the separation of phases (e.g., aqueous and lipids), while allowing them to be mixed.

“Flavoring agent” refers to a substance capable of providing a flavor. In addition to providing a palatable and pleasurable factor to the user, the flavoring agent can also mask undesirable flavors present in the OTF. The flavoring agent can include natural flavoring agents (e.g., extracts)

“Flavor extract” refers to a flavoring agent obtained by extracting a part of a raw material, e.g., animal or plant material, often by using a solvent such as ethanol or water. The majority of natural essences are obtained by extracting the essential oil from the blossoms, fruit, roots, etc., or the whole plants, through four techniques: expression (when the oil is very plentiful and easily obtained, as in lemon peel), absorption (generally accomplished by steeping in alcohol, as vanilla beans), maceration (used to create smaller bits of the whole, as in making peppermint extract, etc.), and distillation (used with maceration, but in many cases, it requires expert chemical knowledge and the erection of costly stills).

Flavoring agents can include breath freshening compounds like menthol, spearmint, and cinnamon, coffee beans, other flavors or fragrances such as fruit (e.g., cherry, orange, grape, etc.) flavors, especially those used for oral hygiene, as well as actives used in dental and oral cleansing such as quaternary ammonium bases. The effect of flavors may be enhanced using flavor enhancers like tartaric acid, citric acid, vanillin, or the like.

As used herein, “sweetener” a substance capable of providing a palatable and pleasurable factor to the user, and/or capable of masking undesirable flavors present in the OTF. The sweetener can include one or more artificial sweeteners, one or more natural sweeteners, or a combination thereof.

Artificial sweeteners include, e.g., acesulfame potassium (available as Nutrinova®), alitame, aspartame (available as NutraSweet® and Equal®), salt of aspartame-acesulfame (available as Twinsweet®), neohesperidin dihydrochalcone, dihydrochalcone compounds, neotame (available as NutraSweet®), sodium cyclamate, saccharin and its various salts such as the sodium salt (available as Sweet'N Low®), stevia, chloro derivatives of sucrose such as sucralose (available as Kaltame® and Splenda®), and mogrosides; 3,6-dihydro-6-methyl-1-1-1,2,3-oxathiazin-4-one-2,2-dioxide, particularly the potassium salt)(acesulfame-K®), sodium and calcium salts thereof.

Natural sweeteners include, e.g., glucose, dextrose, invert sugar, fructose, sucrose, glycyrrhizin; monoammonium glycyrrhizinate (sold under the trade name MagnaSweet®); Stevia Rebaudiana (Stevioside), natural intensive sweeteners, such as Lo Han Kuo, polyols such as sorbitol, mannitol, xylitol, erythritol, and the like.

“Palatable” refers to a substance (e.g., oral thin film) being relatively acceptable or agreeable to the palate or taste (e.g., sweet or savory), and in some cases to the olfactory nerves.

“Dye or pigment” or “coloring agent” refers to a substance that imparts coloring and/or aesthetic appearance to the OTF. A dye is a colored substance that has an affinity to the substrate to which it is being applied. The dye is generally applied in an aqueous solution, and requires a mordant to improve the fastness of the dye on the fiber. A pigment is a material that changes the color of reflected or transmitted light as the result of wavelength-selective absorption. This physical process differs from fluorescence, phosphorescence, and other forms of luminescence, in which a material emits light. Both dyes and pigments appear to be colored because they absorb some wavelengths of light more than others. In contrast with a dye, a pigment generally is insoluble, and has no affinity for the substrate. Some dyes can be precipitated with an inert salt to produce a lake pigment, and based on the salt used they could be aluminum lake, calcium lake or barium lake pigments.

One or more dyes, pigments, and coloring agents can be employed in the manufacture of the thin firm, such that the thin film has the desired color. Suitable colors include, e.g., white, black, yellow, blue, green, pink, red, orange, violet, indigo, and brown. In specific embodiments, the color of the thin film can indicate the contents (e.g., one or more active ingredients) contained therein. For example, the thin film can include one or more sweeteners as indicated by the color of the thin film. Specifically, the thin film can be blue, as an indication that the active ingredient includes aspartame (marketed as Equal®. Alternatively, the thin film can be blue, as an indication that the active ingredient includes sildenafil citrate (marketed as Viagra®). The thin film can be pink, as an indication that the active ingredient includes saccharine. The thin film can be yellow, as an indication that the active ingredient includes sucralose (marketed as Splenda®). Alternatively, the thin film can be yellow, as an indication that the active ingredient includes saccharine (marketed as Sugar) Twin®). The thin film can be green, as an indication that the active ingredient includes stevia. The thin film can be black, as an indication that the active ingredient includes cyclamates. The thin film can be brown, as an indication that the active ingredient includes brown sugar. The thin film can be white, as an indication that the active ingredient includes white sugar.

“Preservative” refers to an agent that extends the storage life of food and nonfood products by retarding or preventing deterioration of flavor, odor, color, texture, appearance, nutritive value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involve mode of action (a preservative prevents growth rather than killing microorganisms) and exposure time (a preservative has days to months to act whereas a sanitizer has at most a few minutes to act).

“Powder coating” refers to a substance that when used on the external surface of an OTF, prevents, minimizes and/or mitigates the likelihood that the OTF will stick to another adjoining OTF once packaged and/or manufacturing equipment. As such, the powder coating can serve as a processing aid. The powder coating can also provide a vehicle for additional flavoring. The size of the substances present in the powder coating can vary as desired, but will typically be in the range of about 1 μm to about 100 μm. In some embodiments, an active ingredient is located in the powder coating. In further embodiments, the powder coating can augment the dissolution rate of the active ingredient located therein.

“Tensile strength” refers to the maximum stress that a material can withstand while being stretched or pulled before failing or breaking. Tensile strength is the opposite of compressive strength and the values can be quite different. Tensile strength is defined as a stress, which is measured as force per unit area. For some non-homogeneous materials (or for assembled components) it can be reported just as a force or as a force per unit width. In the SI system, the unit is the pascal (Pa) (or a multiple thereof, often megapascals (MPa), using the mega-prefix); or, equivalently to pascals, newtons per square meter (N/m2). The customary unit is pounds-force per square inch (lbf/in2 or psi), or kilo-pounds per square inch (ksi, or sometimes kpsi), which is equal to 1000 psi; kilo-pounds per square inch are commonly used for convenience when measuring tensile strengths. Typically, the testing involves taking a small sample with a fixed cross-section area, and then pulling it with a controlled, gradually increasing force until the sample changes shape or breaks.

“Pliable” refers to the ability of an article to readily bend, be flexible, or to be supple.

“Non-sticky” refers to an article (e.g., thin film) not having the property of readily adhering or sticking to another surface (e.g., another article, manufacturing equipment, packaging material, the user, etc.).

“Soft” refers to an article being relatively smooth and agreeable to the touch; not rough or coarse. Such an article will be capable of producing agreeable sensations, pleasant or comfortable, upon contact with an animal such as a human.

“Chewable configuration” refers to an article being manufactured in such a manner and with ingredients, that it possesses a configuration capable of being readily chewed by an animal, such as a human.

“Malleable configuration” refers to refers to an article being manufactured in such a manner and with ingredients, that it possesses a configuration capable of being readily shaped or changed in form (e.g., folded, bent, rolled, twisted, flexed, etc.) without breaking.

“Ductile property” refers to the ability of an article (e.g., thin film) being readily shaped or changed in form (e.g., folded, bent, rolled, twisted, flexed, etc.) without breaking.

“Mixing” refers to the act of combining, uniting, and/or joining multiple substances, into one mass, collection, or assemblage (e.g., slurry), generally with a thorough and continuous contacting of the constituents.

“Blending” refers to the act of mixing that employs equipment typically referred to as a blender, or any device capable of blending a mixture. The mixing can provide a relatively smooth mixture, where the constituents are inseparable. When used in the context of “high shear blending”, the blender has sharp edged blades and is used at high speed (1000-10,000 rpm).

“Mixture” refers to the mass, collection, or assemblage (e.g., slurry) obtained from the act of mixing.

As used herein, “contacting” refers to the act of touching, making contact, or of bringing substances into immediate proximity.

“Heating” refers to the act of applying or transferring a sufficient amount of energy (e.g., thermal energy), within a suitable period of time, such that a rise in temperature is experienced.

“Conductive heat transfer” or “conduction” refers to the transfer of heat from one condensed material into another condensed material that does not involve bulk motion within either of the condensed media.

“Radiative heat transfer” or “radiation” refers to the transfer of heat from one article to another by way of electromagnetic means, usually by infrared radiation, but can also be microwave radiation.

“Convective heat transfer” or “convection” refers to the transfer of heat from one article to another, by the movement of fluids. Convection is usually the dominant form of heat transfer in liquids and gases. Although often discussed as a distinct method of heat transfer, convective heat transfer involves the combined processes of conduction (heat diffusion) and advection (heat transfer by bulk fluid flow).

Convection can be “forced” by movement of a fluid by means other than buoyancy forces (for example, a water pump in an automobile engine). In some cases, natural buoyancy forces alone are entirely responsible for fluid motion when the fluid is heated, and this process is called “natural convection.” An example is the draft in a chimney or around any fire. In natural convection, an increase in temperature produces a reduction in density, which causes fluid motion due to pressures and forces when fluids of different densities are affected by gravity (or any g-force). For example, when water is heated on a stove, hot water from the bottom of the pan rises, displacing the colder denser liquid which falls. After heating has stopped, mixing and conduction from this natural convection eventually result in a nearly homogeneous density, and even temperature.

Two types of convective heat transfer can be distinguished: free or natural convection (passive) and forced convection (active). Active convection occurs when a fluid is forced to flow over the surface by an external source such as fans, by stirring, and pumps, creating an artificially induced convection current. Passive convention occurs when fluid motion is caused by buoyancy forces that result from the density variations due to variations of temperature in the fluid. In the absence of an external source, when the fluid is in contact with a hot surface, its molecules separate and scatter, causing the fluid to be less dense. As a consequence, the fluid is displaced while the cooler fluid gets denser and the fluid sinks. Thus, the hotter volume transfers heat towards the cooler volume of that fluid. Familiar examples are the upward flow of air due to a fire or hot object and the circulation of water in a pot that is heated from below.

As used herein, “washing” refers to the act of removing impurities located on a substrate (e.g., solids) with the use of liquid. In doing so, the impurities located on the substrate will dissolve in the liquid, to subsequently be carried off.

As used herein, “separating” refers to the act of removing, breaking contact, or of removing substances from immediate proximity.

“Packaging material” refers to those materials and substances employed to package the product (e.g., thin film). Such materials are widely known to those of skill in the art.

“Enclosing” refers to the packaging materials containing or holding the product (e.g., thin film) by surrounding the product with the packaging material. The packaging materials can partially surround the product, or can completely surround the product. Typically, to ensure safety (e.g., no tampering with product) and freshness, the packaging materials will completely surround the product. For example, the packaging materials can form a relatively vapor impermeable enclosure of the product.

“Printed indicia” refers to a marking, image, text, and/or symbol located on the surface of the packaging material. The indicia can be placed on the surface of the packaging material by any suitable means (e.g., ink printing, laser printing, etc.). The indicia can include, e.g., a printed message or instructions, list of ingredients (active and inactive), weight of product, manufacturer name and address, manufacturer trademark, etc.

The thin films described herein can be perforated. “Perforated” refers to the one or more holes, apertures or scores existing along a line to facilitate separation. Perforations on the thin films allow the user to conveniently administer smaller dosages of the active ingredient. This is especially useful, for example, when the patient is a child, who should receive a smaller dosage. Accurate dosing can be metered, e.g., by the weight, size, age, etc. of the patient.

“Therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes: (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

The thin film can be administered, e.g., to a human patient in need of a treatment of a disease or disorder. Selection of the active ingredient(s) within the thin film described herein will be dependent upon the disease or disorder to be treated. The above-mentioned references (e.g., Physician's Desk Reference, 2010 Edition) provide a description of the diseases or disorders that specific active ingredients have been approved for by the U.S. FDA, in the marketing and sale of the product within the United States. As such, a skilled artisan can look to such references for guidance in the selection of the active ingredient(s) to be present within the thin film, based upon the treatment of the specific disease or disorder of particular interest.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The active ingredient(s) can be present in any suitable and appropriate amount, depending upon the desired dosing. For example, in a 100 mg thin film, the active ingredient(s) can be present in an amount of about 0.01-60 mg, about 0.1-50 mg, or about 0.5-40 mg.

As used herein, “inorganic acid” or “mineral acid” refers to an acid derived from one or more inorganic compounds. All mineral acids form hydrogen ions and the conjugate base ions when dissolved in water. Commonly used mineral acids include, e.g., hydrochloric acid (HCl), nitric acid (HNO₃), phosphoric acid (H₃PO₄), sulphuric acid (H₂SO₄), boric acid (H₃BO₃), hydrofluoric acid (HF), hydrobromic acid (HBr), and perchloric acid (HClO₄).

As used herein, “organic acid” refers to an organic compound with acidic properties. The most common organic acids are the carboxylic acids, whose acidity is associated with their carboxyl group —COOH. Sulfonic acids, containing the group —SO₂OH, are relatively stronger acids. Alcohols, with —OH, can act as acids but they are usually very weak. The relative stability of the conjugate base of the acid determines its acidity. Other groups can also confer acidity, usually weakly: the thiol group —SH, the enol group, and the phenol group. Commonly used organic acids include, e.g., formic acid, acetic acid, propionic acid, butyric acid, valeric acid, caprioc acid, oxalic acid, lactic acid, malic acid, citric acid, benzoic acid, and carbonic acid.

As used herein, “inorganic base” refers to a base derived from one or more inorganic compounds. All inorganic bases form hydroxyl ions and the conjugate acid ions when dissolved in water. Commonly used inorganic bases include, e.g., sodium hydroxide, potassium hydroxide, and lithium hydroxide.

As used herein, “organic base” refers to an organic compound which acts as a base. Organic bases are usually, but not always, proton acceptors. They usually contain nitrogen atoms, which can easily be protonated. Amines and nitrogen-containing heterocyclic compounds are organic bases. Examples include, e.g., pyridine, methyl amine, imidazole, benzimidazole, histidine, phosphazene bases, and hydroxides of some organic cations.

As used herein, “buffer” refers to a weak acid or base used to maintain the acidity (pH) of a solution near a chosen value after the addition of another acid or base. That is, the function of a buffering agent is to prevent a rapid change in pH when acids or bases are added to the solution. Buffering agents have variable properties—some are more soluble than others; some are acidic while others are basic.

### Additional/Optional Components

A variety of optional components and fillers also may be added to the films. These may include, without limitation: surfactants; plasticizers; polyalcohols; antifoaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors. In some embodiments, more than one active ingredient may be included in the film.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active agent(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers. Inventive films may further include compounds such as butylated hydroxytoluene.

Further additives include inorganic fillers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

Specific plasticizers useful with zein include, e.g., sodium lauryl sulfate, trimethyl citrate, triethyl citrate, triacetyl glycerin, and PEGs (up to 1,100, such as, e.g., 300, 400, 600, 800, and 1,100).

There may further be added compounds to improve the texture and/or flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e., with C₁₂-, C₁₄-, C₁₆-, C₁₈-, C₂₀- and C₂₂-fatty acids.

The total amounts used of the fats, mono-, di-glycerides and/or lecithins may be up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds typically act as flow agents.

Other optional ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols. If desired, the film may include other additives, such as keratin, or proteins, including proteins that are useful in forming a gel, such as gelatine.

Further potential additives include solubility enhancing agents, such as substances that form inclusion compounds with active ingredients. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

### Kits

Pharmaceutical kits are also within the ambit of the present invention. Such kits include a therapeutically effective amount of a thin film as described herein. Sterilization of the thin film and/or packaging material may be carried out using conventional sterilization methodology well-known to those skilled in the art. Instructions or printed indicia, either as inserts or as labels, indicating quantities of the components to be administered, guidelines for administration, may also be included in the kit.

### Utility

The thin films described herein can be useful to deliver a high load of active ingredients to the intended target. The thin film can be placed, e.g., in the mouth thereby administering active ingredients via absorption in the mouth (buccally or sublingually), the stomach (gastrically), and/or via the small intestines (enterically). Such an OTF will be edible (suitable for human consumption), and pharmaceutically acceptable. The thin films can be prepared typically using hydrophilic polymers that rapidly dissolve on the tongue or buccal cavity, delivering the active ingredient to the systemic circulation via dissolution when contact with liquid is made. The thin film can also be used to adhere to mucosal tissue (e.g., at least one of mouth, nose, eye, vagina, and rectum), thereby locally delivering the active ingredient(s) to those bodily tissues. As such, the mucoadhesive films may be used for the administration of an active to any of several body surfaces, especially those including mucous membranes, such as oral, anal, vaginal, opthalmological, the surface of a wound, either on a skin surface or within a body such as during surgery, and similar surfaces.

The thin film can be manufactured to include a relatively high load (e.g., up to about 40 wt. %) of active ingredient. The active ingredient can include active pharmaceutical ingredients (APIs) (e.g., prescription (Rx), over the counter (OTC), and biologicals), veterinary agents, vitamins, neutraceuticals, supplements (e.g., dietary, nutritional, and herbal), and cosmetics. As such, the active ingredients described herein can be useful to treat a disease or disorder typically encountered by the target subject (e.g., human, animal, etc.). Selection of the appropriate active ingredient will influence each of the target subject (e.g., human, animal, etc.), as well as the disease or disorder to be treated. From a practical perspective, selection of both the target subject and the disease or disorder to be treated will influence the selection of the active ingredients that is employed. Furthermore, the ingredients used in construction of the films may be selected to allow for a range of disintegration times for the films.

The films may be applied under or to the tongue of the mammal. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the thin films described herein takes advantage of the films' tendency to dissolve quickly when introduce to a liquid. An active ingredient may be introduced to a liquid (or liquid containing substance) by preparing a film as described herein, introducing it to a liquid (or liquid containing substance), and allowing it to dissolve. This may be used either to prepare a liquid dosage form of an active. This may also be used to flavor a beverage or food product, or to add at least one of a sweetener, electrolytes, nutrients, neutraceuticals, active ingredient, vitamins, and protein to a beverage or food product.

### Active Pharmaceutical Ingredient (API)

As used herein, “active ingredient” refers to a therapeutic agent and includes any substance, other than food, used in the prevention, diagnosis, alleviation, treatment, or cure of a disease or disorder. Stedman's Medical Dictionary, 25th Edition (1990). The substance can be taken by mouth; injected into a muscle, the skin, a blood vessel, or a cavity of the body; or topically applied. Mosby's Medical, Nursing & Allied Health Dictionary, 5th Edition (1998). The agent can include any substance disclosed in at least one of: The Merck Index, 14th Edition (2006); Pei-Show Juo, Concise Dictionary of Biomedicine and Molecular Biology, (1996); U.S. Pharmacopeia Dictionary, 2000 Edition; Physician's Desk Reference, 2010 Edition; Orange Book: Approved Drug Products with Therapeutic Equivalence Evaluations (April 2013); and Approved Animal & Veterinary Drug Products (Green Book) (January 2013). The term active ingredient includes, e.g., prescription and over the counter active pharmaceutical ingredients (e.g., small molecules, macrocycles, peptides, etc.), vitamins, nutraceuticals, supplements (e.g., dietary, nutritional, and herbal), cosmetics, and biologicals.

The compositions disclosed herein, as well as the thin films manufactured from such compositions, can include any suitable active pharmaceutical ingredient (“API”). In various embodiments, advantages of the invention include the encapsulation of the API in the composition, such that unpleasant flavors associated with the API are effectively masked. Such unpleasant flavors can include, e.g., bitterness as well as metallic taste (such as such as Cu, Fe, and/or Zn). As such, specific APIs useful in the present invention include those having an unpleasant taste, such as, for example:
Amodiaquine, which is 4-[(7-chloroquinolin-4-yl)amino]-2-[(diethylamino)methyl]phenol.
Sildenafil, which is 1-[4-ethoxy-3-(6,7-dihydro-1-methyl-7-oxo-3-propyl-1H-pyrazolo[4,3-d]pyrimidin-5-yl) phenylsulfonyl]-4-methylpiperazine.
Aspirin or acetylsalicylic acid, which is 2-(acetoxy)benzoic acid.
Caffeine, which is 1,3,7-trimethylpurine-2,6-dione.
Ibuprofen, which is (RS)-2-(4-(2-methylpropyl)phenyl)propanoic acid.
Artesunate, which is (3R,5aS,6R,8aS,9R,10S,12R,12aR)-Decahydro-3,6,9-trimethyl-3,12-epoxy-12H-pyrano[4,3-j]-1,2-benzodioxepin-10-ol, hydrogen succinate.
Nicotine, which is (S)-3-[1-Methylpyrrolidin-2-yl]pyridine.
Ranitidine, which is N-(2-[(5-[(dimethylamino)methyl]furan-2-yl)methylthio]ethyl)-N′-methyl-2-nitroethene-1,1-diamine; dimethyl [(5-{[(2-{[1-(methylamino)-2-nitroethenyl]amino}ethyl)sulfanyl]methyl}furan-2-yl)methyl]amine.
Loratidine, which is Ethyl 4-(8-chloro-5,6-dihydro-11H-benzo[5,6]cyclohepta[1,2-b]pyridin-11-ylidene)-1-piperidinecarboxylate.
Loperamide, which is 4-[4-(4-Chlorophenyl)-4-hydroxypiperidin-1-yl]-N,N-dimethyl-2,2-diphenylbutanamide.

### Methods of Manufacturing an Encapsulated API

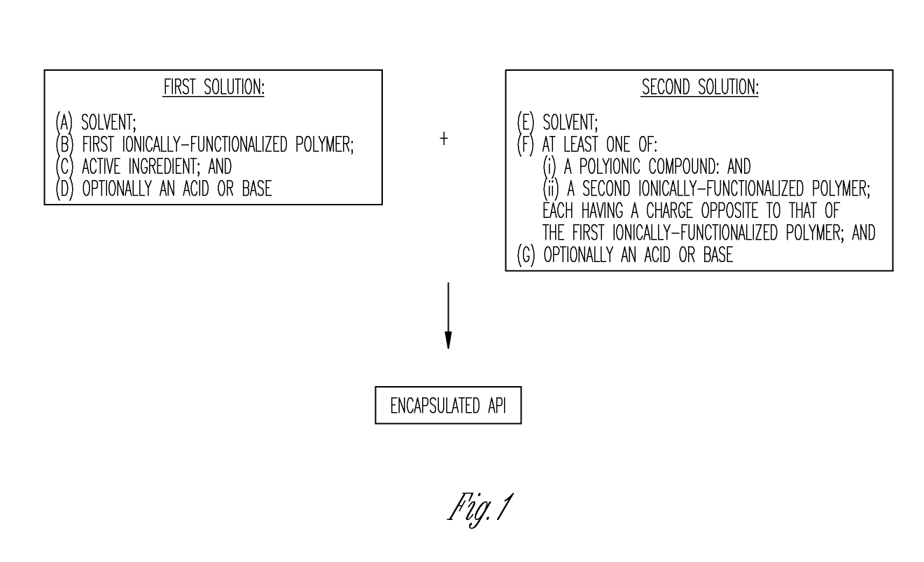

Referring to FIGS. 1-4, methods of manufacturing an encapsulated active pharmaceutical ingredient are provided. Briefly stated (see FIG. 1), a first solution and second solution are contacted, to provide the encapsulated API. The first solution includes solvent, a first ionically-functionalized polymer, active ingredient, and optionally an acid or base. The second solution includes solvent, at least one of: (i) polyionic compound, and (ii) a second ionically-functionized polymer, each having a charge opposite to that of the first ionically-functionalized polymer; and optionally an acid or base.

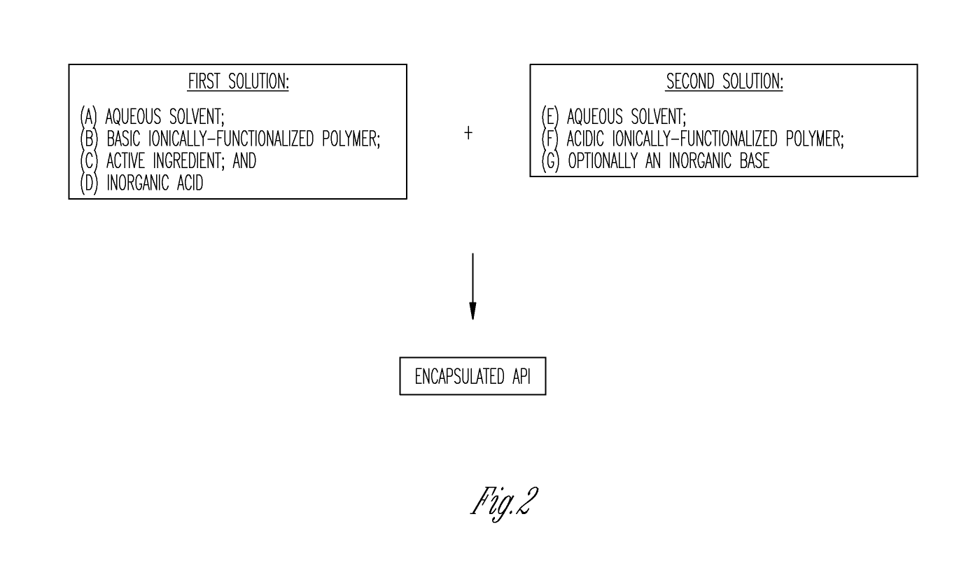

Specifically (see FIG. 2), an aqueous solvent, basic ionically-functionalized polymer, API and inorganic acid are contacted to form the first solution. An aqueous solvent, acidic ionically-functionized polymer and optionally an organic base are contacted to form the second solution. The first solution and second solution are contacted, to provide the encapsulated API.

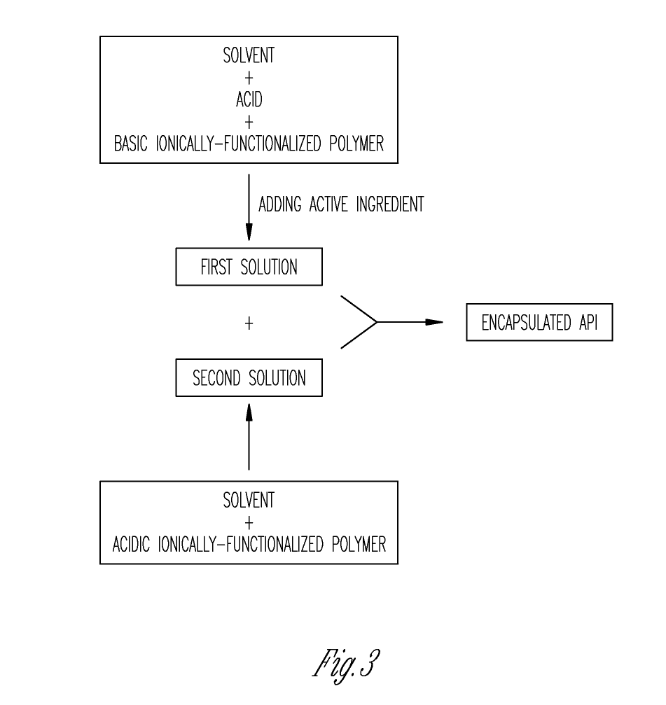

Specifically (see FIG. 3), to the solvent, acidic and basic ionically-functionalized polymer is added the API, to form the first solution. Solvent and acidic ionically-functionalized polymer are contacted, to form the second solution. The first solution and second solution are contacted, to provide the encapsulated API.

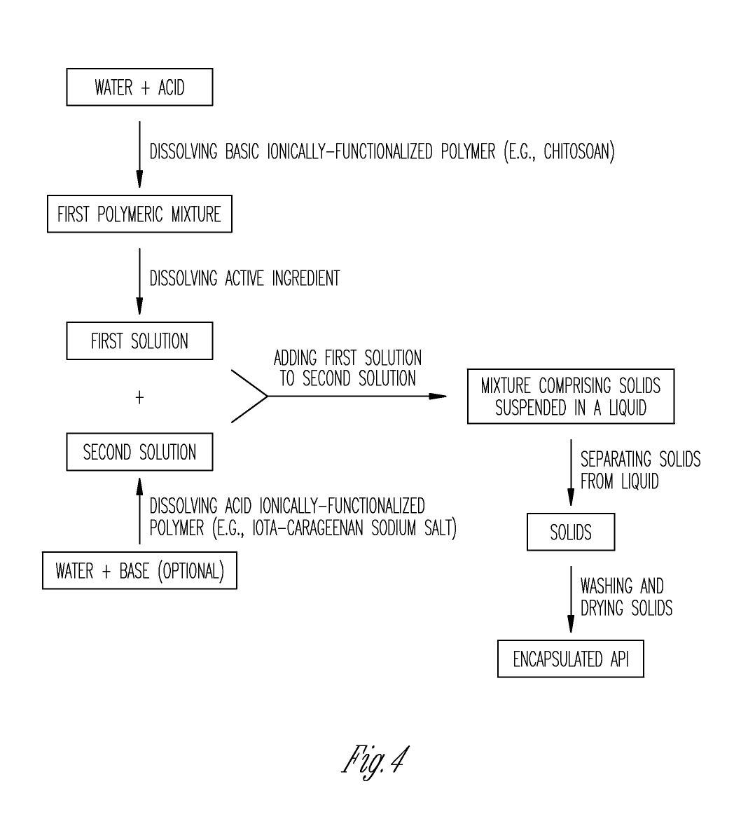

Specifically (see FIG. 4), water and acid are contacted, and a basic ionically-functionalized polymer (e.g., chitosan) is added, to form a first polymeric mixture. An API is dissolved in the first polymeric mixture, to provide a first solution. An acidic ionically-functionalized polymer (e.g., iota-carrageenan) is dissolved in water and base (optional), to provide a second solution. The first solution is added to the second solution, to provide a mixture that includes solids suspended in a liquid. The solids are separated from the liquid, washed and dried, to provide the encapsulated API.

Additional descriptions are illustrated in the Examples herein.

### Methods of Manufacturing a Thin Film

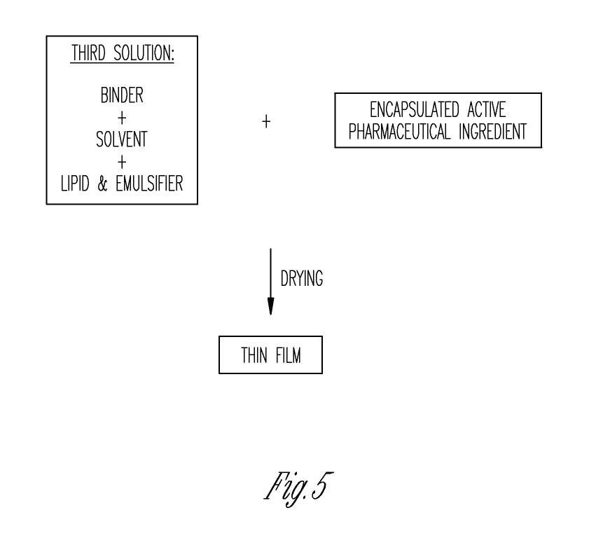

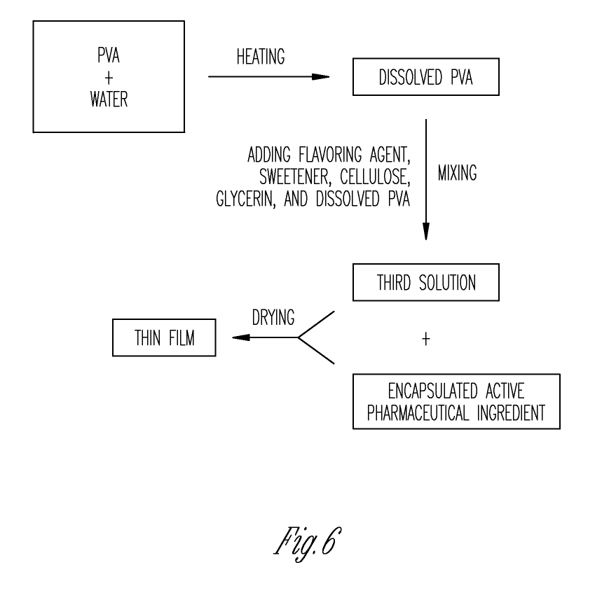

Referring to FIGS. 5-6, methods of manufacturing thin films are provided. Briefly stated (see FIG. 5), a third solution that includes binder, solvent and lipid & emulsifier are contacted with an encapsulated API. This mixture is dried to provide the thin film.

Specifically (see FIG. 6), water and PVA is heated, sufficient to dissolve the PVA. To this mixture is added flavoring agent, sweetener, cellulose, glycerin and dissolved PVA, to provide a third solution. The third solution is contacted with an encapsulated API and dried to provide the thin film.

Additional descriptions are illustrated in the Examples herein.

### Enumerated Embodiments

Specific enumerated embodiments [1] to [117] provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

[1.] The present invention provides a composition that includes:

(a) a first ionically-functionalized polymer;

(b) at least one of (i) a polyionic compound and (ii) a second ionically-functionalized polymer, each independently having a charge opposite to that of the first ionically-functionalized polymer;

(c) solvent; and

(d) active ingredient.

[2.] The present invention also provides the composition of the above embodiment, wherein the active ingredient is at least partially encapsulated.
[3.] The present invention also provides the composition of any one of the above embodiments, further including at least one of an inorganic acid, an organic acid, an inorganic base, an organic base, and a buffer.
[4.] The present invention also provides the composition of any one of the above embodiments, wherein the ionically-functionalized polymer includes one or more basic polymers, one or more acidic polymers, or a combination thereof.
[5.] The present invention also provides the composition of any one of the above embodiments, wherein the ionically-functionalized polymer includes about 1-3 ionic functionalities per monomer.
[6.] The present invention also provides the composition of any one of the above embodiments, wherein the basic ionically-functionalized polymer includes one or more amino (—NH₂) groups, one or more quaternary ammonium cations (—NH₃⁺), or a combination thereof.
[7.] The present invention also provides the composition of any one of the above embodiments, wherein the basic ionically-functionalized polymer includes at least one of: one or more secondary ammonium groups, one or more tertiary ammonium groups, and one or more quaternary ammonium groups.
[8.] The present invention also provides the composition of any one of the above embodiments, wherein the basic ionically-functionalized polymer includes at least one of zein, chitosan and polyquaternium.
[9.] The present invention also provides the composition of any one of the above embodiments, wherein the acidic ionically-functionalized polymer includes one or more carboxylic acid (—CO₂H) groups, one or more sulfate (—OSO₃H) groups, one or more sulfonate (—SO₃H) groups, one or more phosphate (—OPO₃H₂) groups, one or more phosphonate (—PO₃H₂) groups, or a combination thereof.
[10.] The present invention also provides the composition of any one of the above embodiments, wherein the acidic ionically-functionalized polymer includes one or more of pectin, xanthan gum, careageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya.
[11.] The present invention also provides the composition of any one of the above embodiments, wherein the polyionic compound includes one or more acidic non-polymeric crosslinkers.
[12.] The present invention also provides the composition of any one of the above embodiments, wherein the polyionic compound includes about 2-5 ionic functionalities per monomer.
[13.] The present invention also provides the composition of any one of the above embodiments, wherein the acidic polyionic compound includes one or more carboxylic acid or ester (—CO₂H) groups, one or more phosphate (—OPO₃H₂) groups, or a combination thereof.
[14.] The present invention also provides the composition of any one of the above embodiments, wherein the polyionic compound is a polyanionic compound.
[15.] The present invention also provides the composition of any one of the above embodiments, wherein the polyionic compound includes at least one of citric acid, sodium triphosphate, malonic acid, malic acid, fumaric acid, succinic acid, glutaric acid, adipic acid, pimelic acid, and suberic acid.
[16.] The present invention also provides the composition of any one of the above embodiments, wherein the composition includes:

a first solution that includes a basic ionically-functionalized polymer and solvent; and

a second solution that includes an acidic ionically-functionalized polymer and solvent.

[17.] The present invention also provides the composition of any one of the above embodiments, wherein the composition includes:

a first solution including a basic ionically-functionalized polymer, optionally an acid, and solvent; and

a second solution including an acidic ionically-functionalized polymer, optionally a base, and solvent.

[18.] The present invention also provides the composition of any one of the above embodiments, wherein the composition includes:

a first solution that includes chitosan, optionally an acid, active ingredient, and solvent; and

a second solution including:

at least one of pectin, xanthan gum, carrageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya;

optionally a base; and

solvent.

[19.] The present invention also provides the composition of any one of the above embodiments, wherein the first ionically-functionalized polymer is at least partially ionically crosslinked to at least one of the second ionically-functionalized polymer and polyionic compound.
[20.] The present invention also provides the composition of any one of the above embodiments, wherein the first ionically-functionalized polymer is at least partially ionically crosslinked to at least one of the second ionically-functionalized polymer and polyionic compound, such that the crosslinking provides a matrix that at least partially encapsulates the active ingredient.
[21.] The present invention also provides the composition of any one of the above embodiments, wherein the first ionically-functionalized polymer is at least partially ionically crosslinked to at least one of the second ionically-functionalized polymer and polyionic compound, such that the crosslinking provides a matrix wherein the active ingredient is dispersed within the first ionically crosslinked polymer and at least one of the second ionically-functionalized polymer and the polyionic compound.
[22.] The present invention also provides the composition of any one of the above embodiments, wherein the first ionically-functionalized polymer is at least partially ionically crosslinked to at least one of the second ionically-functionalized polymer and polyionic compound, such that the crosslinking provides a matrix wherein the active ingredient is uniformly dispersed within the first ionically crosslinked polymer and at least one of the second ionically-functionalized polymer and the polyionic compound.
[23.] The present invention also provides the composition of any one of the above embodiments, wherein the first ionically-functionalized polymer is at least partially ionically crosslinked to at least one of the second ionically-functionalized polymer and polyionic compound, such that the crosslinking provides an active ingredient surrounded by a shell that includes the first ionically crosslinked polymer and at least one of the second ionically-functionalized polymer and the polyionic compound.
[24.] The present invention also provides the composition of any one of the above embodiments, that includes:

(A) a first solution that includes:
(i) solvent;
(ii) basic ionically-functionalized polymer;
(iii) active ingredient; and
(iv) optionally an acid; and

(B) a second solution that includes:
(i) solvent;
(ii) acidic ionically-functionalized polymer; and
(iii) optionally a base.
   [25.] The present invention also provides the composition of any one of the above embodiments, that includes:

(A) a first solution that includes:
(i) aqueous solvent;
(ii) basic ionically-functionalized polymer;
(iii) active ingredient; and
(iv) inorganic acid; and

(B) a second solution that includes:
(i) aqueous solvent;
(ii) acidic ionically-functionalized polymer; and
(iii) optionally an inorganic base.
   [26.] The present invention also provides the composition of any one of the above embodiments, which is a thin film configured for application to at least one of a mouth, buccal cavity, nose, eye, vagina, and rectum.
   [27.] The present invention also provides the composition of any one of the above embodiments, wherein the active ingredient is present in at least about 20 wt. %.
   [28.] The present invention also provides the composition of any one of the above embodiments, wherein the active ingredient is present in up to about 90 wt. %.
   [29.] The present invention also provides the composition of any one of the above embodiments, further including at least one of:

(a) lipid & emulsifier,

(b) sweetener,

(c) flavoring agent,

(d) binder, and

(e) coloring agent.

[30.] The present invention also provides the composition of embodiment [29], wherein the binder includes at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, carboxymethyl cellulose (CMC), and polyethylene glycol.
[31.] The present invention also provides the composition of embodiment [29], wherein the lipid & emulsifier includes at least one of glycerin, propylene glycol, and polyethylene glycol.
[32.] The present invention also provides the composition of any one of the above embodiments, wherein the first ionically-functionalized polymer is at least partially crosslinked to at least one of the polyionic compound and the second ionically-functionalized polymer.
[33.] The present invention also provides a thin film manufactured from the composition of any one of the above embodiments.
[34.] The present invention also provides a thin film manufactured from the composition of any one of the above embodiments, wherein at least about 50 wt. %, in the aggregate, of the one or more active ingredients is encapsulated.
[35.] The present invention also provides a thin film manufactured from the composition of any one of the above embodiments, wherein up to about 90 wt. %, in the aggregate, of the one or more active ingredients is encapsulated.
[36.] The present invention also provides a thin film manufactured from the composition of any one of the above embodiments, wherein the one or more active ingredients is at least partially encapsulated.
[37.] The present invention also provides a thin film manufactured from the composition of any one of the above embodiments, wherein the one or more active ingredients is completely encapsulated.
[38.] The present invention also provides a thin film manufactured from the composition of any one of the above embodiments, wherein the one or more active ingredients, in the aggregate, are present in at least about 35 wt. %.
[39.] The present invention also provides a thin film manufactured from the composition of any one of the above embodiments, wherein the one or more active ingredients, in the aggregate, are present in up to about 75 wt. %.
[40.] The present invention also provides a thin film manufactured from the composition of any one of the above embodiments, further including a preservative, present in about 0-0.02 wt. %.
[41.] The present invention also provides a thin film manufactured from the composition of any one of the above embodiments, further including a powder coating.
[42.] The present invention also provides a thin film manufactured from the composition of any one of the above embodiments, which is palatable to a human.
[43.] The present invention also provides a thin film manufactured from the composition of any one of the above embodiments, wherein the external surfaces have a smooth texture.
[44.] The present invention also provides a thin film manufactured from the composition of any one of the above embodiments, which has a high tensile strength.
[45.] The present invention also provides a thin film manufactured from the composition of any one of the above embodiments, which is pliable.
[46.] The present invention also provides a thin film manufactured from the composition of any one of the above embodiments, which is non-sticky to touch.
[47.] The present invention also provides a thin film manufactured from the composition of any one of the above embodiments, which does not readily stick to another thin film.
[48.] The present invention also provides a thin film manufactured from the composition of any one of the above embodiments, which is relatively soft to touch.
[49.] The present invention also provides a thin film manufactured from the composition of any one of the above embodiments, having a chewable configuration.
[50.] The present invention also provides a thin film manufactured from the composition of any one of the above embodiments, having a resilient configuration.
[51.] The present invention also provides a thin film manufactured from the composition of any one of the above embodiments, having an elastic or malleable configuration.
[52.] The present invention also provides a thin film manufactured from the composition of any one of the above embodiments, having a ductile property.
[53.] The present invention also provides a method of preparing an encapsulated active pharmaceutical ingredient, the method includes contacting a first solution that includes:

(a) solvent;

(b) first ionically-functionalized polymer;

(c) active ingredient; and

(d) optionally an acid or base;

with a second solution that includes:

(e) solvent;

(f) at least one of (i) a polyionic compound and (ii) a second ionically-functionalized polymer, each having a charge opposite to that of the first ionically-functionalized polymer; and

(g) optionally an acid or base.

[54.] The present invention also provides a method of preparing an encapsulated active pharmaceutical ingredient, the method includes contacting a first solution that includes:

(a) aqueous solvent;

(b) basic ionically-functionalized polymer;

(c) active ingredient; and

(d) inorganic acid; and

with a second solution that includes:

(e) aqueous solvent;

(f) acidic ionically-functionalized polymer; and

(g) optionally an inorganic base.

[55.] The present invention also provides a method of any one of the above embodiments, that includes:

(A) forming a first solution that includes:
(i) contacting a solvent, acid, and basic ionically-functionalized polymer to form a first polymeric mixture;
(ii) contacting an active ingredient and the first polymeric mixture, to form a first solution;

(B) forming a second solution that includes:
(iii) contacting a solvent and an acidic ionically-functionalized polymer, to form a second solution; and

(C) contacting the first solution and the second solution.

[56.] The present invention also provides a method of embodiment [55], wherein in step (A)(i), acid, water and basic ionically-functionalized polymer are stirred and heated to up to about 80° C.
[57.] The present invention also provides a method of any one of embodiments [55]-[56], wherein in step (A)(i), the basic ionically-functionalized polymer is added to a mixture of the acid and solvent.
[58.] The present invention also provides a method of any one of embodiments [55]-[57], wherein in step (A)(ii), the active ingredient is added to the first polymeric mixture.
[59.] The present invention also provides a method of any one of embodiments [55]-[58], wherein in step (B)(iii), iota-carrageenan sodium salt is added to water, to form the second solution.
[60.] The present invention also provides a method of any one of embodiments [55]-[59], wherein in step (B)(iii), iota-carrageenan sodium salt is added to water, and heated to a temperature of up to about 50° C., to form the second solution.
[61.] The present invention also provides a method of any one of embodiments [55]-[60], wherein in step (B)(iii), the second solution is formed from the solvent, the acidic ionically-functionalized polymer, and a base.
[62.] The present invention also provides a method of any one of embodiments [55]-[61], wherein in step (C), the first solution is added to the second solution.
[63.] The present invention also provides a method of any one of embodiments [55]-[62], wherein in step (C), the first solution is added to the second solution, while rapidly blending or mixing.
[64.] The present invention also provides a method of any one of embodiments [55]-[63], wherein in step (C), the first solution is slowly added to the second solution, drop-wise or in a stream.
[65.] The present invention also provides a method of any one of embodiments [55]-[64], wherein the product obtained in step (C) is a mixture includes solids suspended in a liquid.
[66.] The present invention also provides a method of any one of embodiments [55]-[65], wherein the product obtained in step (C) is a mixture that includes solids suspended in a liquid, the method further including separating the solids from the liquid.
[67.] The present invention also provides a method of embodiment [66], further including washing the solids.
[68.] The present invention also provides a method of embodiment [67], further including suspending the solids in a solvent and separating the solids from the solvent.
[69.] The present invention also provides a method of any one of embodiments [55]-[68], further including drying the product obtained therein to form an encapsulated active pharmaceutical ingredient.
[70.] The present invention also provides a method of any one of the above embodiments, that includes:

(A) forming a first solution that includes:
(i) dissolving chitosan in a mixture of water and acid, to form a first polymeric mixture;
(ii) dissolving an active ingredient in the first polymeric mixture, to form the first solution;

(B) forming a second solution that includes:
(iii) dissolving iota-carrageenan sodium salt in water and optionally a base, to form the second solution; and

(C) adding the first solution to the second solution, while blending or mixing, to form a mixture including solids suspended in a liquid;

(D) separating the solids from the liquid; and

(E) washing and drying the solids.

[71.] The present invention also provides a method of any one of the above embodiments, further including:

contacting a binder, solvent and lipid & emulsifier, to form a third solution,

contacting the third solution with the encapsulated active pharmaceutical ingredient, and

drying, to provide a thin film.

[72.] The present invention also provides a method of embodiment [71], wherein the contacting of the binder, solvent and lipid & emulsifier, to form a third solution, includes:

mixing PVA in water and heating,

adding flavoring agent, sweetener, cellulose, glycerin, and dissolved PVA, and mixing,

adding the encapsulated active pharmaceutical ingredient, and mixing, and

drying.

[73.] The present invention also provides a thin film that includes:

(a) a first ionically-functionalized polymer;

(b) at least one of (i) a polyionic compound and (ii) a second ionically-functionalized polymer, each independently having a charge opposite to that of the first ionically-functionalized polymer;

(c) solvent;

(d) binder,

(e) lipid & emulsifier, and

(f) active ingredient.

[74.] The present invention also provides a thin film of the above embodiment, further including at least one of:

(a) sweetener,

(b) flavoring agent,

(c) binder, and

(d) coloring agent.

[75.] The present invention also provides a thin film of any one of the above embodiments, wherein the first ionically-functionalized polymer is at least partially crosslinked to at least one of the polyionic compound and the second ionically-functionalized polymer.
[76.] The present invention also provides a thin film of any one of the above embodiments, wherein the first ionically-functionalized polymer includes one or more basic polymers, one or more acidic polymers, or a combination thereof.
[77.] The present invention also provides a thin film of any one of the above embodiments, wherein the first ionically-functionalized polymer includes about 1-3 ionic functionalities per monomer.
[78.] The present invention also provides a thin film of any one of the above embodiments, wherein the second ionically-functionalized polymer includes one or more amino (—NH₂) groups, one or more quaternary ammonium cations (—NH₃⁺), or a combination thereof.
[79.] The present invention also provides a thin film of any one of the above embodiments, wherein the second ionically-functionalized polymer includes at least one of: one or more secondary ammonium groups, one or more tertiary ammonium groups, and one or more quaternary ammonium groups.
[80.] The present invention also provides a thin film of any one of the above embodiments, wherein the second ionically-functionalized polymer includes at least one of zein, chitosan and polyquaternium.
[81.] The present invention also provides a thin film of any one of the above embodiments, wherein the first ionically-functionalized polymer includes one or more carboxylic acid (—CO₂H) groups, one or more sulfate (—OSO₃H) groups, one or more sulfonate (—SO₃H) groups, one or more phosphate (—OPO₃H₂) groups, one or more phosphonate (—PO₃H₂) groups, or a combination thereof.
[82.] The present invention also provides a thin film of any one of the above embodiments, wherein the first ionically-functionalized polymer includes one or more of pectin, xanthan gum, careageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya.
[83.] The present invention also provides a thin film of any one of the above embodiments, wherein the polyionic compound includes one or more acidic non-polymeric crosslinkers.
[84.] The present invention also provides a thin film of any one of the above embodiments, wherein the polyionic compound includes about 2-5 ionic functionalities per monomer.
[85.] The present invention also provides a thin film of any one of the above embodiments, wherein the polyionic compound includes one or more carboxylic acid or ester (—CO₂H) groups, one or more phosphate (—OPO₃H₂) groups, or a combination thereof.
[86.] The present invention also provides a thin film of any one of the above embodiments, wherein the polyionic compound is a polyanionic compound.
[87.] The present invention also provides a thin film of any one of the above embodiments, wherein the polyionic compound includes at least one of citric acid, sodium triphosphate, malonic acid, malic acid, fumaric acid, succinic acid, glutaric acid, adipic acid, pimelic acid, and suberic acid.
[88.] The present invention also provides a thin film of any one of the above embodiments, wherein the first ionically-functionalized polymer is at least partially ionically crosslinked to at least one of the second ionically-functionalized polymer and polyionic compound, such that the crosslinking provides a matrix that at least partially encapsulates the active ingredient.
[89.] The present invention also provides a thin film of any one of the above embodiments, wherein the first ionically-functionalized polymer is at least partially ionically crosslinked to at least one of the second ionically-functionalized polymer and polyionic compound, such that the crosslinking provides a matrix wherein the active ingredient is dispersed within the first ionically crosslinked polymer and at least one of the second ionically-functionalized polymer and the polyionic compound.
[90.] The present invention also provides a thin film of any one of the above embodiments, wherein the first ionically-functionalized polymer is at least partially ionically crosslinked to at least one of the second ionically-functionalized polymer and polyionic compound, such that the crosslinking provides a matrix wherein the active ingredient is uniformly dispersed within the first ionically crosslinked polymer and at least one of the second ionically-functionalized polymer and the polyionic compound.
[91.] The present invention also provides a thin film of any one of the above embodiments, wherein the first ionically-functionalized polymer is at least partially ionically crosslinked to at least one of the second ionically-functionalized polymer and polyionic compound, such that the crosslinking provides an active ingredient surrounded by a shell including the first ionically crosslinked polymer and at least one of the second ionically-functionalized polymer and the polyionic compound.
[92.] The present invention also provides a thin film of any one of the above embodiments, which is a thin film configured for application to at least one of a mouth, buccal cavity, nose, eye, vagina, and rectum.
[93.] The present invention also provides a thin film of any one of the above embodiments, wherein the active ingredient is present in at least about 20 wt. %.
[94.] The present invention also provides a thin film of any one of the above embodiments, wherein the active ingredient is present in up to about 90 wt. %.
[95.] The present invention also provides a thin film of any one of the above embodiments, wherein the active ingredient is present in at least about 35 wt. %.
[96.] The present invention also provides a thin film of any one of the above embodiments, wherein the active ingredient is present in up to about 75 wt. %.
[97.] The present invention also provides a thin film of any one of the above embodiments, further including a binder, selected from at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, carboxymethyl cellulose (CMC), and polyethylene glycol.
[98.] The present invention also provides a thin film of any one of the above embodiments, further including a lipid & emulsifier, selected from at least one of glycerin, propylene glycol, and polyethylene glycol.
[99.] The present invention also provides a thin film of any one of the above embodiments, wherein the first ionically-functionalized polymer is at least partially crosslinked to at least one of the polyionic compound and the second ionically-functionalized polymer.
[100.] The present invention also provides a thin film of any one of the above embodiments, wherein at least about 50 wt. % of the active ingredient is encapsulated.
[101.] The present invention also provides a thin film of any one of the above embodiments, wherein up to about 90 wt. % of the active ingredient is encapsulated.
[102.] The present invention also provides a thin film of any one of the above embodiments, wherein the active ingredient is at least partially encapsulated.
[103.] The present invention also provides a thin film of any one of the above embodiments, wherein the active ingredient is completely encapsulated.
[104.] The present invention also provides a thin film of any one of the above embodiments, further including a preservative, present in about 0-0.02 wt. %.
[105.] The present invention also provides a thin film of any one of the above embodiments, further including a powder coating.
[106.] The present invention also provides a thin film of any one of the above embodiments, which is palatable to a human.
[107.] The present invention also provides a thin film of any one of the above embodiments, wherein the external surfaces have a smooth texture.
[108.] The present invention also provides a thin film of any one of the above embodiments, which has a high tensile strength.
[109.] The present invention also provides a thin film of any one of the above embodiments, which is pliable.
[110.] The present invention also provides a thin film of any one of the above embodiments, which is non-sticky to touch.
[111.] The present invention also provides a thin film of any one of the above embodiments, which does not readily stick to another thin film.
[112.] The present invention also provides a thin film of any one of the above embodiments, which is relatively soft to touch.
[113.] The present invention also provides a thin film of any one of the above embodiments, having a chewable configuration.
[114.] The present invention also provides a thin film of any one of the above embodiments, having a resilient configuration.
[115.] The present invention also provides a thin film of any one of the above embodiments, having an elastic or malleable configuration.
[116.] The present invention also provides a thin film of any one of the above embodiments, having a ductile property.
[117]. The present invention also provides a thin film of any one of the above embodiments, wherein the active pharmaceutical ingredient includes at least one of:
Amodiaquine, which is 4-[(7-chloroquinolin-4-yl)amino]-2-[(diethylamino)methyl]phenol;
Sildenafil, which is 1-[4-ethoxy-3-(6,7-dihydro-1-methyl-7-oxo-3-propyl-1H-pyrazolo[4,3-d]pyrimidin-5-yl) phenylsulfonyl]-4-methylpiperazine;
Aspirin or acetylsalicylic acid, which is 2-(acetoxy)benzoic acid;
Caffeine, which is 1,3,7-trimethylpurine-2,6-dione;
Ibuprofen, which is (RS)-2-(4-(2-methylpropyl)phenyl)propanoic acid;
Artesunate, which is (3R,5aS,6R,8aS,9R,10S,12R,12aR)-Decahydro-3,6,9-trimethyl-3,12-epoxy-12H-pyrano[4,3-j]-1,2-benzodioxepin-10-ol, hydrogen succinate;
Nicotine, which is (S)-3-[1-Methylpyrrolidin-2-yl]pyridine;
Ranitidine, which is N-(2-[(5-[(dimethylamino)methyl]furan-2-yl)methylthio]ethyl)-N′-methyl-2-nitroethene-1,1-diamine; dimethyl [(5-{[(2-{[1-(methylamino)-2-nitroethenyl]amino}ethyl)sulfanyl]methyl}furan-2-yl)methyl]amine;
Loratidine, which is Ethyl 4-(8-chloro-5,6-dihydro-11H-benzo[5,6]cyclohepta[1,2-b]pyridin-11-ylidene)-1-piperidinecarboxylate; and
Loperamide, which is 4-[4-(4-Chlorophenyl)-4-hydroxypiperidin-1-yl]-N,N-dimethyl-2,2-diphenylbutanamide.

### EXAMPLES

### Example 1: Encapsulated Artesunate-Amodiaquine Strip

| Material | Brand | Quantity | % Non-Vol |
|---|---|---|---|
| H₂O | | mL | 0.0 |
| Lipids & Emulsifiers | | | |
| Glycerin | | 8.00 g | 8.0 |
| Sweeteners | | | |
| Sucralose | | 6.00 g | 6.0 |
| Acesulfame Potassium | | 3.00 g | 3.0 |
| (ACE-K) | | | |
| MagnaSweet MM100 | | 0.40 g | 0.40 |
| Flavoring agents | | | |
| Cherry Powder Flavoring | Virginia | 2.00 g | 2.0 |
| (CA93) | Dare | | |
| Blackberry Powder Flavoring | | 2.00 g | 2.0 |
| (BV93) | | | |
| Malic Acid | | 1.00 g | 1.0 |
| NaCl | | 0.75 g | 0.75 |
| Binders | | | |
| Microcrystalline Cellulose | | 4.00 g | 4.0 |
| Pectin | | 6.00 g | 6.0 |
| PVA | Spectrum | 22.3 g | 22.3 |
| | P1180, USP | | |
| | Grade | | |
| Actives | | | |
| Amodiaquine, encapsulated, | | 318.1 g | 30.19 AQE |
| 77.8% loading | | | |
| Artesunate, encapsulated, | | 110.6 g | 14.38 ASE |
| 60.47% loading | | | |
| Coloring agents | | | |
| FD&C Red #40 | | | |
| Total | | | 100 |

Mixed PVA in 120 mL H₂O with heating for ^{˜}1 hour, stir/heat plate setting 3 for heat. Turned off heat and stirred overnight. Mixed all the flavors, sweeteners, cellulose, glycerin, and dissolved PVA with a high shear mixer in a 400 mL beaker. Poured this into a blender with a low shear mixer blade. Added the encapsulated amodiaquine and encapsulated artesunate+60 mL H₂O. Mixed thoroughly and poured onto a glass sheet and cooked at 70° C. for ^{˜}30 minutes. This was a little dry, so set plate out to rehydrate slightly over the weekend.

Ran the remainder of the material on the new ASI (Advanced Systems, Inc.) oven in the factory, 93° C. at 1 ft/min, extruded at 1.4 mm thickness. The final product peels off the PET, may be slightly too dry at these conditions. Put product in a bag to age over the weekend.

HPLC analysis of the lab-made strip showed 11.4% Artesunate and 32.3% Amodiaquine.2H₂O.2HCl. In a 287.4 mg strip, the artesunate is 32.7 mg, and (0.323)(287.4 mg)((355.861)/(464.814))=71.1 mg.

### Example 2: Chitosan, i-Carrageenan, Amodiaquine 80% Loading

| | MW | | | Wt. | Mol | |
|---|---|---|---|---|---|---|
| Ingredient | (g/mol) | Quantity | | Equiv. | Equiv. | mmol |
| Solution 1 | | | | | | |
| Water | | 350 | mL | | | |
| HCl, 1.000 N | 36.46 | 49.6 | mL | | 1.00 | 49.6 |
| Chitosan | 161.16 | 8.00 | g | 2.00 | 1.00 | 49.6 |
| Amodiaquine | 464.81 | 78.37 | g | | 3.40 | 168.60 |
| dihydrochloride | (355.86 | (equiv. to | | | | |
| dihydrate | free | 60.0 | g | | | |
| | base) | free base) | | | | |
| Solution 2 | | | | | | |
| Water | | 400 | mL | | | |
| Iota-Carrageenan | 255.19 | 4.00 | g | 1.00 | 0.32 | 15.68 |
| Sodium Hydroxide | 40.00 | 15.47 | g | | 7.80 | 386.8 |

Iota-Carrageenan disaccharide monomer is C₁₂H₁₆O₁₅S₂.2Na, has 2 acid functionalities each as a sodium salt (Kelco type TPC-1), and mw=510.36. MW/acid functionality is half that, or 255.19. Protonated the MW/acid functionality is 211.23.

Mixed Solution 1 with a magnetic stirring rod with heating (heat setting 3.0 on stir plate). Solution became clear and orange after about 45 minutes. Mixed Solution 2 with magnetic stirrer with no heating for about 1 hour. Put Solution 2 into an ice-cream mixer, which gave a folding action an no-shear. I dripped Solution 1 into Solution 2 using a 500 mL separatory funnel. Solid material formed during the entire addition. Collected the solids using a centrifuge, ^{˜}2-4 minutes per run. Resuspended the solid in water and centrifuged again. Dried a quantity of wet solid material in a 50 mL beaker then placed the beaker in a 100° C. oven for 1.5 hours. Dried to a constant weight. Wet material is 12.2% solid material 87.8% water. Loading is 77.8%.

HPLC analysis showed the loading of the wet material to be 12.4% as amodiaquine.2HCl.2H₂O, or 9.49%=(12.4%)(355.861 mw amodiaquine)/(464.814 amodiaquine.2HCl2H₂O) of the amodiaquine anhydrous base. Loading is 77.8%=((0.124)/(0.122))((355.861)/(464.814)). Theoretical loading is 9.76%=(12.2% solid in water)(80.0% loading).

### Example 3: Chitosan, i-Carrageenan, Artesunate 80% Loading

| | MW | | | Wt. | Mol | |
|---|---|---|---|---|---|---|
| Ingredient | (g/mol) | Quantity | | Equiv. | Equiv. | mmol |
| Solution 1 | | | | | | |
| Water | | 220 | mL | | | |
| HCl, 1.000 N | 36.46 | 31 | mL | | 1.00 | 31.03 |
| Chitosan | 161.16 | 5.00 | g | 2.00 | 1.00 | 31.03 |
| Artesunate | 384.42 | 30.00 | g | 12.00 | | 78.04 |
| Solution 2 | | | | | | |
| Water | | 125 | mL | | | |
| i-Carrageenan | 255.19 | 2.50 | g | 1.00 | | 9.80 |

ι-Carrageenan disaccharide monomer is C₁₂H₁₆O₁₅S₂.2Na, has 2 acid functionalities each as a sodium salt (Kelco type TPC-1), and mw=510.36. MW/acid functionality is half that, or 255.19. Protonated the MW/acid functionality is 211.23.

Mixed water, HCl, and chitosan to make Solution 1 with a magnetic stirring rod with heating (heat setting 3.0 on stir plate). Solution became clear after about 45 minutes. Cooled with stirring in a crystallization dish filled with water and ice. Added the artesunate and mixed with a high-shear hand mixer. Mixed Solution 2 at RT overnight with magnetic stirrer. Put Solution 2 into a blender with a low-shear blade. Dripped Solution 1 into Solution 2. The solution thickened drastically then thinned toward the end of the addition. Solid material formed during the entire addition. Collected the solids using a clinical centrifuge, ^{˜}170 mL at a time, each set spinning for about 5 minutes. Resuspended the solid in about 500 mL water then collected it by re-centrifugation. Placed 967 mg of wet solid material in a 50 mL beaker then placed the beaker in a 100° C. oven for 1.5 hours. Dried to a constant weight. Obtained 208 mg dry encapsulated Artesunate. Wet material is 21.5% solid material 78.5% water.

HPLC analysis showed the loading of the wet material to be 13.0%. Theoretical loading is 17.2%=(21.5% solid in water)(80% loading).

What is claimed is:

1. A composition comprising:
(a) a first ionically-functionalized polymer;
(b) at least one of (i) a polyionic compound and (ii) a second ionically-functionalized polymer, each independently having a charge opposite to that of the first ionically-functionalized polymer;
(c) solvent; and
(d) active ingredient.

2. The composition of claim 1, wherein the active ingredient is at least partially encapsulated.

3. The composition of claim 1, further comprising at least one of an inorganic acid, an organic acid, an inorganic base, an organic base, and a buffer.

4. The composition of claim 1, wherein the ionically-functionalized polymer comprises one or more basic polymers, one or more acidic polymers, or a combination thereof.

5. The composition of claim 1, wherein the ionically-functionalized polymer comprises about 1-3 ionic functionalities per monomer.

6. The composition of claim 1, wherein the basic ionically-functionalized polymer comprises one or more amino (—NH₂) groups, one or more quaternary ammonium cations (—NH₃⁺), or a combination thereof.

7. The composition of claim 1, wherein the basic ionically-functionalized polymer comprises at least one of: one or more secondary ammonium groups, one or more tertiary ammonium groups, and one or more quaternary ammonium groups.

8. The composition of claim 1, wherein the basic ionically-functionalized polymer comprises at least one of zein, chitosan and polyquaternium.

9. The composition of claim 1, wherein the acidic ionically-functionalized polymer comprises one or more carboxylic acid (—CO₂H) groups, one or more sulfate (—OSO₃H) groups, one or more sulfonate (—SO₃H) groups, one or more phosphate (—OPO₃H₂) groups, one or more phosphonate (—PO₃H₂) groups, or a combination thereof.

10. The composition of claim 1, wherein the acidic ionically-functionalized polymer comprises one or more of pectin, xanthan gum, careageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya.

11. The composition of claim 1, wherein the polyionic compound comprises one or more acidic non-polymeric crosslinkers.

12. The composition of claim 1, wherein the polyionic compound comprises about 2-5 ionic functionalities per monomer.

13. The composition of claim 1, wherein the acidic polyionic compound comprises one or more carboxylic acid or ester (—CO₂H) groups, one or more phosphate (—OPO₃H₂) groups, or a combination thereof.

14. The composition of claim 1, wherein the polyionic compound is a polyanionic compound.

15. The composition of claim 1, wherein the polyionic compound comprises at least one of citric acid, sodium triphosphate, malonic acid, malic acid, fumaric acid, succinic acid, glutaric acid, adipic acid, pimelic acid, and suberic acid.

16. The composition of claim 1, wherein the composition comprises:
a first solution comprising a basic ionically-functionalized polymer and solvent; and
a second solution comprising an acidic ionically-functionalized polymer and solvent.

17. The composition of claim 1, wherein the composition comprises:
a first solution comprising a basic ionically-functionalized polymer, optionally an acid, and solvent; and
a second solution comprising an acidic ionically-functionalized polymer, optionally a base, and solvent.

18. The composition of claim 1, wherein the composition comprises:
a first solution comprising chitosan, optionally an acid, active ingredient, and solvent; and
a second solution comprising:
at least one of pectin, xanthan gum, carrageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya;
optionally a base; and
solvent.

19. The composition of claim 1, wherein the first ionically-functionalized polymer is at least partially ionically crosslinked to at least one of the second ionically-functionalized polymer and polyionic compound.

20. The composition of claim 1, wherein the first ionically-functionalized polymer is at least partially ionically crosslinked to at least one of the second ionically-functionalized polymer and polyionic compound, such that the crosslinking provides a matrix that at least partially encapsulates the active ingredient.

21. The composition of claim 1, wherein the first ionically-functionalized polymer is at least partially ionically crosslinked to at least one of the second ionically-functionalized polymer and polyionic compound, such that the crosslinking provides a matrix wherein the active ingredient is dispersed within the first ionically crosslinked polymer and at least one of the second ionically-functionalized polymer and the polyionic compound.

22. The composition of claim 1, wherein the first ionically-functionalized polymer is at least partially ionically crosslinked to at least one of the second ionically-functionalized polymer and polyionic compound, such that the crosslinking provides a matrix wherein the active ingredient is uniformly dispersed within the first ionically crosslinked polymer and at least one of the second ionically-functionalized polymer and the polyionic compound.

23. The composition of claim 1, wherein the first ionically-functionalized polymer is at least partially ionically crosslinked to at least one of the second ionically-functionalized polymer and polyionic compound, such that the crosslinking provides an active ingredient surrounded by a shell comprising the first ionically crosslinked polymer and at least one of the second ionically-functionalized polymer and the polyionic compound.

24. A composition comprising:
(A) a first solution comprising:
(i) solvent;
(ii) basic ionically-functionalized polymer;
(iii) active ingredient; and
(iv) optionally an acid; and
(B) a second solution comprising:
(i) solvent;
(ii) acidic ionically-functionalized polymer; and
(iii) optionally a base.

25. A composition comprising:
(A) a first solution comprising:
(i) aqueous solvent;
(ii) basic ionically-functionalized polymer;
(iii) active ingredient; and
(iv) inorganic acid; and
(B) a second solution comprising:
(i) aqueous solvent;
(ii) acidic ionically-functionalized polymer; and
(iii) optionally an inorganic base.

26. The composition of claim 1, which is a thin film configured for application to at least one of a mouth, buccal cavity, nose, eye, vagina, and rectum.

27. The composition of claim 1, wherein the active ingredient is present in at least about 20 wt. %.

28. The composition of claim 1, wherein the active ingredient is present in up to about 90 wt. %.

29. The composition of claim 1, further comprising at least one of:
(a) lipid & emulsifier,
(b) sweetener,
(c) flavoring agent,
(d) binder, and
(e) coloring agent.

30. The composition of claim 29, wherein the binder comprises at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, carboxymethyl cellulose (CMC), and polyethylene glycol.

31. The composition of claim 29, wherein the lipid & emulsifier comprises at least one of glycerin, propylene glycol, and polyethylene glycol.

32. The composition of claim 1, wherein the first ionically-functionalized polymer is at least partially crosslinked to at least one of the polyionic compound and the second ionically-functionalized polymer.

33. A thin film manufactured from the composition of any one of claims 1-32.

34. A thin film manufactured from the composition of any one of claims 1-32, wherein at least about 50 wt. %, in the aggregate, of the one or more active ingredients is encapsulated.

35. A thin film manufactured from the composition of any one of claims 1-32, wherein up to about 90 wt. %, in the aggregate, of the one or more active ingredients is encapsulated.

36. A thin film manufactured from the composition of any one of claims 1-32, wherein the one or more active ingredients is at least partially encapsulated.

37. A thin film manufactured from the composition of any one of claims 1-32, wherein the one or more active ingredients is completely encapsulated.

38. A thin film manufactured from the composition of any one of claims 1-32, wherein the one or more active ingredients, in the aggregate, are present in at least about 35 wt. %.

39. A thin film manufactured from the composition of any one of claims 1-32, wherein the one or more active ingredients, in the aggregate, are present in up to about 75 wt. %.

40. A thin film manufactured from the composition of any one of claims 1-32, further including a preservative, present in about 0-0.02 wt. %.

41. A thin film manufactured from the composition of any one of claims 1-32, further including a powder coating.

42. A thin film manufactured from the composition of any one of claims 1-32, which is palatable to a human.

43. A thin film manufactured from the composition of any one of claims 1-32, wherein the external surfaces have a smooth texture.

44. A thin film manufactured from the composition of any one of claims 1-32, which has a high tensile strength.

45. A thin film manufactured from the composition of any one of claims 1-32, which is pliable.

46. A thin film manufactured from the composition of any one of claims 1-32, which is non-sticky to touch.

47. A thin film manufactured from the composition of any one of claims 1-32, which does not readily stick to another thin film.

48. A thin film manufactured from the composition of any one of claims 1-32, which is relatively soft to touch.

49. A thin film manufactured from the composition of any one of claims 1-32, having a chewable configuration.

50. A thin film manufactured from the composition of any one of claims 1-32, having a resilient configuration.

51. A thin film manufactured from the composition of any one of claims 1-32, having an elastic or malleable configuration.

52. A thin film manufactured from the composition of any one of claims 1-32, having a ductile property.

53. A method of preparing an encapsulated active pharmaceutical ingredient, the method comprising contacting a first solution comprising:
(a) solvent;
(b) first ionically-functionalized polymer;
(c) active ingredient; and
(d) optionally an acid or base;
with a second solution comprising:
(e) solvent;
(f) at least one of (i) a polyionic compound and (ii) a second ionically-functionalized polymer, each having a charge opposite to that of the first ionically-functionalized polymer; and
(g) optionally an acid or base.

54. A method of preparing an encapsulated active pharmaceutical ingredient, the method comprising contacting a first solution comprising:
(a) aqueous solvent;
(b) basic ionically-functionalized polymer;
(c) active ingredient; and
(d) inorganic acid; and
with a second solution comprising:
(e) aqueous solvent;
(f) acidic ionically-functionalized polymer; and
(g) optionally an inorganic base.

55. A method comprising:
(A) forming a first solution comprising:
(i) contacting a solvent, acid, and basic ionically-functionalized polymer to form a first polymeric mixture;
(ii) contacting an active ingredient and the first polymeric mixture, to form a first solution;
(B) forming a second solution comprising:
(iii) contacting a solvent and an acidic ionically-functionalized polymer, to form a second solution; and
(C) contacting the first solution and the second solution.

56. The method of claim 55, wherein in step (A)(i), acid, water and basic ionically-functionalized polymer are stirred and heated to up to about 80° C.

57. The method of claim 55, wherein in step (A)(i), the basic ionically-functionalized polymer is added to a mixture of the acid and solvent.

58. The method of claim 55, wherein in step (A)(ii), the active ingredient is added to the first polymeric mixture.

59. The method of claim 55, wherein in step (B)(iii), iota-carrageenan sodium salt is added to water, to form the second solution.

60. The method of claim 55, wherein in step (B)(iii), iota-carrageenan sodium salt is added to water, and heated to a temperature of up to about 50° C., to form the second solution.

61. The method of claim 55, wherein in step (B)(iii), the second solution is formed from the solvent, the acidic ionically-functionalized polymer, and a base.

62. The method of claim 55, wherein in step (C), the first solution is added to the second solution.

63. The method of claim 55, wherein in step (C), the first solution is added to the second solution, while rapidly blending or mixing.

64. The method of claim 55, wherein in step (C), the first solution is slowly added to the second solution, drop-wise or in a stream.

65. The method of claim 55, wherein the product obtained in step (C) is a mixture comprising solids suspended in a liquid.

66. The method of claim 55, wherein the product obtained in step (C) is a mixture comprising solids suspended in a liquid, the method further comprising separating the solids from the liquid.

67. The method of claim 66, further comprising washing the solids.

68. The method of claim 67, further comprising suspending the solids in a solvent and separating the solids from the solvent.

69. The method of claim 55, further comprising drying the product obtained therein to form an encapsulated active pharmaceutical ingredient.

70. A method comprising:
(A) forming a first solution comprising:
(i) dissolving chitosan in a mixture of water and acid, to form a first polymeric mixture;
(ii) dissolving an active ingredient in the first polymeric mixture, to form the first solution;
(B) forming a second solution comprising:
(iii) dissolving iota-carrageenan sodium salt in water and optionally a base, to form the second solution; and
(C) adding the first solution to the second solution, while blending or mixing, to form a mixture comprising solids suspended in a liquid;
(D) separating the solids from the liquid; and
(E) washing and drying the solids.

71. The method of claim 70, further comprising:
contacting a binder, solvent and lipid & emulsifier, to form a third solution,
contacting the third solution with the encapsulated active pharmaceutical ingredient, and
drying, to provide a thin film.

72. The method of claim 71, wherein the contacting of the binder, solvent and lipid & emulsifier, to form a third solution, comprises:
mixing PVA in water and heating,
adding flavoring agent, sweetener, cellulose, glycerin, and dissolved PVA, and mixing,
adding the encapsulated active pharmaceutical ingredient, and mixing, and drying.

73. A thin film comprising:
(a) a first ionically-functionalized polymer;
(b) at least one of (i) a polyionic compound and (ii) a second ionically-functionalized polymer, each independently having a charge opposite to that of the first ionically-functionalized polymer;
(c) solvent;
(d) binder,
(e) lipid & emulsifier, and
(f) active ingredient.

74. The thin film of the claim 73, further comprising at least one of:
(a) sweetener,
(b) flavoring agent,
(c) binder,
(d) coloring agent, and
(e) lipid & emulsifier.

75. The thin film of claim 73, wherein the first ionically-functionalized polymer is at least partially crosslinked to at least one of the polyionic compound and the second ionically-functionalized polymer.

76. The thin film of claim 73, wherein the first ionically-functionalized polymer comprises one or more basic polymers, one or more acidic polymers, or a combination thereof.

77. The thin film of claim 73, wherein the first ionically-functionalized polymer comprises about 1-3 ionic functionalities per monomer.

78. The thin film of claim 73, wherein the second ionically-functionalized polymer comprises one or more amino (—NH₂) groups, one or more quaternary ammonium cations (—NH₃⁺), or a combination thereof.

79. The thin film of claim 73, wherein the second ionically-functionalized polymer comprises at least one of: one or more secondary ammonium groups, one or more tertiary ammonium groups, and one or more quaternary ammonium groups.

80. The thin film of claim 73, wherein the second ionically-functionalized polymer comprises at least one of zein, chitosan and polyquaternium.

81. The thin film of claim 73, wherein the first ionically-functionalized polymer comprises one or more carboxylic acid (—CO₂H) groups, one or more sulfate (—OSO₃H) groups, one or more sulfonate (—SO₃H) groups, one or more phosphate (—OPO₃H₂) groups, one or more phosphonate (—PO₃H₂) groups, or a combination thereof.

82. The thin film of claim 73, wherein the first ionically-functionalized polymer comprises one or more of pectin, xanthan gum, careageenan, gellan gum, carbomer, carboxymethylcellulose, carboxymethyl starch, crosscarmellose, gamma-polyglutamic acid, welan gum, alginic acid, diutan gum, hyaluronic acid, chondroitin sulfate, alguronic acid, and gum karaya.

83. The thin film of claim 73, wherein the polyionic compound comprises one or more acidic non-polymeric crosslinkers.

84. The thin film of claim 73, wherein the polyionic compound comprises about 2-5 ionic functionalities per monomer.

85. The thin film of claim 73, wherein the polyionic compound comprises one or more carboxylic acid or ester (—CO₂H) groups, one or more phosphate (—OPO₃H₂) groups, or a combination thereof.

86. The thin film of claim 73, wherein the polyionic compound is a polyanionic compound.

87. The thin film of claim 73, wherein the polyionic compound comprises at least one of citric acid, sodium triphosphate, malonic acid, malic acid, fumaric acid, succinic acid, glutaric acid, adipic acid, pimelic acid, and suberic acid.

88. The thin film of claim 73, wherein the first ionically-functionalized polymer is at least partially ionically crosslinked to at least one of the second ionically-functionalized polymer and polyionic compound, such that the crosslinking provides a matrix that at least partially encapsulates the active ingredient.

89. The thin film of claim 73, wherein the first ionically-functionalized polymer is at least partially ionically crosslinked to at least one of the second ionically-functionalized polymer and polyionic compound, such that the crosslinking provides a matrix wherein the active ingredient is dispersed within the first ionically crosslinked polymer and at least one of the second ionically-functionalized polymer and the polyionic compound.

90. The thin film of claim 73, wherein the first ionically-functionalized polymer is at least partially ionically crosslinked to at least one of the second ionically-functionalized polymer and polyionic compound, such that the crosslinking provides a matrix wherein the active ingredient is uniformly dispersed within the first ionically crosslinked polymer and at least one of the second ionically-functionalized polymer and the polyionic compound.

91. The thin film of claim 73, wherein the first ionically-functionalized polymer is at least partially ionically crosslinked to at least one of the second ionically-functionalized polymer and polyionic compound, such that the crosslinking provides an active ingredient surrounded by a shell comprising the first ionically crosslinked polymer and at least one of the second ionically-functionalized polymer and the polyionic compound.

92. The thin film of claim 73, which is a thin film configured for application to at least one of a mouth, buccal cavity, nose, eye, vagina, and rectum.

93. The thin film of claim 73, wherein the active ingredient is present in at least about 20 wt. %.

94. The thin film of claim 73, wherein the active ingredient is present in up to about 90 wt. %.

95. The thin film of claim 73, wherein the active ingredient is present in at least about 35 wt. %.

96. The thin film of claim 73, wherein the active ingredient is present in up to about 75 wt. %.

97. The thin film of claim 73, further comprising a binder, selected from at least one of pectin, microcrystalline cellulose, xanthan gum, locust bean gum, guar gum, gum arabic, gum tragacanth, gum karaya, beta glucan, glucomannan, tapioca starch, carrageenan, xanthan gum, gellan gum, alginic acid or sodium alginate, konjac gum, tara gum, chitosan, agar, maltodextrin, polyvinyl alcohol, pullulan, polycarbophil, povidone, hydroxypropyl methylcellulose, hydroxypropyl cellulose, hydroxypropyl starch, carboxymethyl cellulose (CMC), and polyethylene glycol.

98. The thin film of claim 73, further comprising a lipid & emulsifier, selected from at least one of glycerin, propylene glycol, and polyethylene glycol.

99. The thin film of claim 73, wherein the first ionically-functionalized polymer is at least partially crosslinked to at least one of the polyionic compound and the second ionically-functionalized polymer.

100. The thin film of claim 73, wherein at least about 50 wt. % of the active ingredient is encapsulated.

101. The thin film of claim 73, wherein up to about 90 wt. % of the active ingredient is encapsulated.

102. The thin film of claim 73, wherein the active ingredient is at least partially encapsulated.

103. The thin film of claim 73, wherein the active ingredient is completely encapsulated.

104. The thin film of claim 73, further including a preservative, present in about 0-0.02 wt. %.

105. The thin film of claim 73, further including a powder coating.

106. The thin film of claim 73, which is palatable to a human.

107. The thin film of claim 73, wherein the external surfaces have a smooth texture.

108. The thin film of claim 73, which has a high tensile strength.

109. The thin film of claim 73, which is pliable.

110. The thin film of claim 73, which is non-sticky to touch.

111. The thin film of claim 73, which does not readily stick to another thin film.

112. The thin film of claim 73, which is relatively soft to touch.

113. The thin film of claim 73, having a chewable configuration.

114. The thin film of claim 73, having a resilient configuration.

115. The thin film of claim 73, having an elastic or malleable configuration.

116. The thin film of claim 73, having a ductile property.

117. The thin film of any one of claims 73-116, wherein the active pharmaceutical ingredient comprises at least one of:
Amodiaquine, which is 4-[(7-chloroquinolin-4-yl)amino]-2-[(diethylamino)methyl]phenol;
Sildenafil, which is 1-[4-ethoxy-3-(6,7-dihydro-1-methyl-7-oxo-3-propyl-1H-pyrazolo[4,3-d]pyrimidin-5-yl) phenylsulfonyl]-4-methylpiperazine;
Aspirin or acetylsalicylic acid, which is 2-(acetoxy)benzoic acid;
Caffeine, which is 1,3,7-trimethylpurine-2,6-dione;
Ibuprofen, which is (RS)-2-(4-(2-methylpropyl)phenyl)propanoic acid;
Artesunate, which is (3R,5a5,6R,8a5,9R,10S,12R,12aR)-Decahydro-3,6,9-trimethyl-3,12-epoxy-12H-pyrano[4,3-j]-1,2-benzodioxepin-10-ol, hydrogen succinate;
Nicotine, which is (S)-3-[1-Methylpyrrolidin-2-yl]pyridine;
Ranitidine, which is N-(2-[(5-[(dimethylamino)methyl]furan-2-yl)methylthio]ethyl)-N′-methyl-2-nitroethene-1,1-diamine; dimethyl [(5-{[(2-{[1-(methylamino)-2-nitroethenyl]amino}ethyl)sulfanyl]methyl}furan-2-yl)methyl]amine;
Loratidine, which is Ethyl 4-(8-chloro-5,6-dihydro-11H-benzo[5,6]cyclohepta[1,2-b]pyridin-11-ylidene)-1-piperidinecarboxylate; and
Loperamide, which is 4-[4-(4-Chlorophenyl)-4-hydroxypiperidin-1-yl]-N,N-dimethyl-2,2-diphenylbutanamide.
